(19) United States
(12) Patent Application Publication
(10) Pub. No.: US 2002/0124855 A1
(43) Pub. Date: Sep. 12, 2002

(54) Method of providing a dynamic cellular cardiac support

(76) Inventor: Juan C. Chachques, Paris (FR)

Correspondence Address: HODGSON RUSS LLP, ONE M&T PLAZA, SUITE 2000, BUFFALO, NY, 14203-2391, US

(21) Appl. No.: 10/095,630

(22) Filed: Mar. 11, 2002

Related U.S. Application Data
(60) Provisional application No. 60/274,990, filed on Mar. 12, 2001.

(51) Int. Cl. A61B 19/00

(52) U.S. Cl. 128/898

(57) ABSTRACT

The present invention provides a method for repairing damaged myocardium. The method comprises using a combination of cellular cardiomyoplasty and electrostimulation to synchronize the contractions of the transplanted cells with the cardiac cells. The method comprises the steps of obtaining myogenic cells from a donor and implanting the myogenic cells into the damaged myocardium. Electrical stimulation can be applied to the cells in vitro during culturing of the cells, after implantation of the cells into the myocardium or both.

### FIELD OF THE INVENTION

The present invention relates generally to the field of myocardial repair and more particularly to a method for the repair of damaged myocardium by a combination of cellular based therapy and electrostimulation.

### BACKGROUND OF THE INVENTION

Heart failure is a significant public health problem in contemporary cardiology. Heart failure, estimated to occur in 1% and 4% of the population, increases exponentially with age, so that current demographic trends in industrialized nations predict an increase in the number of patients with heart failure during coming decades as the populations of these countries grow older.

Heart failure is associated with significant morbidity, a high incidence of complications, frequent hospitalization, and rising healthcare costs. Mortality and morbidity caused by cardiac insufficiency are increasing at a time when the overall cardiovascular death rate is on the decline. In the United States alone, an estimated 5 million individuals have a diagnosis of “congestive heart failure”, and an additional 400,000-500,000 new cases are diagnosed annually.

Due to the restricted number of heart donors for heart transplantation and the high cost and drawbacks of mechanical assist devices, a large proportion of the end stage heart failure patients need a therapeutic approach other than the current standard modalities. Approximately 25% of patients included in waiting lists for heart transplantation die, due to the limited donor availability.

Although the use of pharmacological therapies has improved survival in heart failure patients, and new mechanical assist devices and xenotransplantation approaches are currently being developed, mortality remains high, with more than 50% of all patients succumbing within 5 years of initial diagnosis.

Congestive cardiac failure is caused by a decrease in myocardial contractility due to mechanical overload or by an initial defect in the myocardial fiber. The alteration in diastolic function is inextricably linked with the pathophysiology of cardiac insufficiency. Despite a widely varying and diverse etiology of congestive cardiac failure (e.g. ischemic or idiopathic dilated cardiomyopathies), the pathophysiology is to a great extent constant. The predominant factor is the alteration of myocardial contractility. This contractility defect causes an elevation of the ventricular wall tension resulting in a progressive decline in the contractile state of the myocardial fibers.

Congestive heart failure (CHF) is a common medical condition that affects more than 22 million people worldwide. Heart failure develops slowly as the heart muscle weakens and is unable to pump enough blood to meet the body's needs. Weakening of the heart muscle is a result of damage such as from heart attack or coronary artery disease, or from strain placed on the heart by years of untreated high blood pressure, valvular disease, cardiomyopathy or diabetes. A less-efficient, weakened heart must work harder to pump blood to the body and brain.

Heart failure involves in many cases defects of the heart conduction system as well as depressed myocardium contractility together with enlarged ventricular cavities. Heart failure patient death is either due to pump failure or to arrhythmia. Heart attack and heart failure can result in conduction abnormalities such as heart block and conduction delay. Approximate one-third of those people with NYHA Class III/IV heart failure exhibit asynchronous heart rythmn.

Electrical dyssynchronization between chambers (left or right bundle-branch block) are often found in heart failure population. Recent studies aimed at correcting these conduction defects by right atrial/left ventricular or right atrial/biventricular pacing have shown beneficial clinical effects of these pacing modalities. Thus, multisite cardiac pacing to restore appropriate timing between cardiac chambers activities is becoming a valid therapeutic alternative for heart failure patients. However, studies have shown that many patients (up to 40%) experience refractory heart failure due to a persistent myocardial dysfunction, one or two years following the initiation of multisite pacing.

The cellular basis for congestive heart failure centers upon a lack of stem cells in the myocardium and the consequent inability of damaged heart cells to undergo repair or divide. Cellular cardiomyoplasty, (i.e., transplantation of cells) instead of an entire organ, has a number of attractive attributes and is dependent on an ever expanding understanding of the molecular basis of skeletal myogenesis.

Cell transplantation strategies have been designed to replace damaged myocardial cells with cells that can perform cardiac work. The cellular cardiomyoplasty procedure consists of transplanting myogenic cells, such as cultured satellite cells (myoblasts), originating from a skeletal muscle biopsy of leg or arms of the same individual, to the damaged myocardium. Satellite cells are mononucleated cells situated between the sarcolemma and the basal lamina of differentiated muscle fibers. They are thought to be responsible for postnatal growth, muscle fiber repair and regeneration. Another approach for cellular cardiomyoplasty consists of the utilization of bone marrow stem cells, autologous or fetal cardiomyocytes, or smooth muscle cells.

One of the problems limiting hemodynamic benefits of cellular cardiomyoplasty is that, even if the myoblasts survive after implantation, functionally those cells do not contract spontaneously and hence, they do not contribute to improve regional myocardial contractility.

Additionally, following cell transplantation, many cells organize in myotubes, and some differentiate in muscle fibers. In this new cytoarchitectural configuration, a special role is played by contracting proteins (actin and myosin). Because only myotubes displaying contractile activity express slow myosin, there is a need to induce predominant expression of slow fatigue resistant myosin, instead of fast myosin, to improve the development of myotubes. The present invention addresses this need with electrical stimulation.

Electrical Stimulation

Electrical activation of skeletal muscle has important clinical applications. At present, these applied principles are found in the treatment of respiratory paralysis, (stimulation of the diaphragm, or more precisely the phrenic nerve), in colorectal surgery (surgical creation of a neo-sphincter using the gracilis muscle, electrostimulation of this muscle allows an efficient continence), in urology (vesicomyoplasty and neosphincter), in the correction of scolioses (stimulation of the paravertebrae muscles), in the treatment of paraplegia (stimulation of the lower limb muscles), and in orthopaedics (rehabilitation of the musculature after prolonged inactivation).

In cardiology, functional electrostimulation of skeletal muscles has been used to assist ventricular function. The cardiomyoplasty surgical procedure involves the use of an autologous muscle in the form of the latissimus dorsi muscle flap around the ventricles and electrostimulated in a rhythmic fashion during systole. The biological explanation for the success of this operation is physiological adaptation of skeletal muscle induced by chronic muscular electrostimulation enabling it to perform cardiac work (“myocardisation” of the latissimus dorsi muscle). Biochemically, there is a metabolic transformation of rapid muscular fibers with glycolytic metabolism, into slow fibers with oxidative metabolism resistant to fatigue.

Electrostimulation has also been applied in cellular biology studies. In vitro, chronic electrostimulation on myotube cultures derived from neonatal rat hindlimb muscles showed that the expression of adult fiber type-specific fast and slow myosins could be induced by stimulation with appropriate fast- or slow-type stimulus patterns. Electrostimulation of myotube cultures established from satellite cells enhanced the mRNA and protein expression of a developmental isoform of slow myosin (MHCI).

In spite of these studies, there still remains a need to improve cardiac myoplasty techniques so as to enhance the efficiency of such techniques.

### SUMMARY OF THE INVENTION

The present invention provides a method for repairing damaged myocardium. The method comprises using a combination of cellular cardiomyoplasty and electrostimulation to synchronize the contractions of the transplanted cells with the cardiac cells. To perform the method of the present invention, myogenic cells are obtained from a suitable source and implanted into the damaged myocardium. Following implantation, electrical stimulation is applied to facilitate synchronization of the transplanted cells.

In one embodiment, electrical pacing of the myogenic cells is carried out after implantation into the host myocardium. A pacemaker already implanted in the host can be used for this purpose.

In another embodiment, myogenic cells are implanted into the host myocardium after electrical stimulation of the myogenic cells in vitro

In yet another embodiment, myogenic cell cultures are electrically stimulated and implanted into the myocardium. Following implantation, pacing is applied to the implanted myogenic cells.

### DETAILED DESCRIPTION OF THE INVENTION

The present invention provides a method of repairing damaged myocardium by using a combination of cell transplantation and electrical stimulation.

For the present invention, myogenic cells can be obtained from any suitable source. Myogenic cells may be any type of contractile cells including skeletal myoblasts including satellite cells, bone marrow stromal cells, peripheral blood stem cells, post natal marrow mesodermal progenitor cells, smooth muscle cells, adult cardiomyocytes, fetal cardiomyocytes, neonatal cardiomyocytes, embryonic stem cells, various cell lines, bone marrow derived angioblasts, endothelial cells, endothelial progenitor cells, or combinations thereof. The myogenic cells selected for transplantation should be able to differentiate into muscle cells either before or following implantation into the damaged myocardium. In a preferred embodiment, the cells are autologous to reduce the immune response. Thus, in the case of autologous implantation, the myogenic cells are obtained from an individual, cultured and implanted back into the myocardium of the same individual. If the cells are from a non-autologous source, immmunosuppressants may be administered to the recipient.

Following isolation from a suitable source, the myogenic cells may be implanted fresh or may be expanded and/or purified in culture. Methods for expanding and purifying various cell types are well known to those skilled in the art. For example, details of such methods can be obtained from U.S. Pat. Nos. 5,130,141, 6,110,459, and 5,602,301.

The myogenic cells are then implanted into the damaged myocardium. The myogenic cells may be supplemented with various growth factors including, but not limited to, vascular endothelial growth factors (VEGF), fibroblast growth factors (FGFs).

Implantation of the myogenic cells can be accomplished by standard techniques such as via a catheter or direct injection, classic or minimally invasive thorascopic surgical techniques known to those skilled in the art. As described above, the myogenic cell compositions may comprise of cells in suitable implantation solutions, cells in combination with a porous carrier or other implantation components known to those skilled in the art.

Following implantation, electrical stimulation is applied to facilitate synchronization of the transplanted cells. Electrical stimulation of the heart such as in cardiac resynchronization (also known as multisite cardiac pacing) is well known in the field of cardiology. For example, following implantation, atrial synchronized biventricular pacing can be performed. For pacing of transplanted cells, 3 electrodes were implanted for cell pacing and cardiac resynchronisation: 1—Endocardial right atrium electrode. 2—Endocardial right ventricle electrode. 3—Left ventricular electrode were placed into a cardiac vein via the coronary sinus. Chronic atrial synchronized biventricular pacing is performed starting immediately after surgery using a three-chamber cardiac pacemaker. Ventricular channels are programmed using a minimum pulse amplitude of 5 Volts and a pulse width of 0.5 milliseconds. Ventricular electrostimulation included depolarization of the implanted cells. In cases of anatomical vein difficulty, a platinum-iridium epicardial pacing lead can be implanted in the left ventricular wall during cell implantation. Standard pacemakers including the new generation of 3-chamber pacemakers can be used for this invention. In many cases, these pulse generators are already implanted clinically in patients and therefore demonstrated to be safe without substantial risk of induction of malignant ventricular arrythmias or ventricular fibrillation.

Cardiac resynchronization therapy involves atrial-synchronized, simultaneous bi-ventricular pacing i.e., it synchronizes atrioventricular contractons and coordinates ventricular contractions, to alleviate symptoms of patients suffering from CHF by correcting ventricular dysynchrony—the two lower chambers of the heart are not beating together as they do normally. Cardiac resynchronization therapy involves the implant of a system to help the two sides of the heart beat together again and improve its efficiency and increase blood flow to the body (such systems include the Medtronic InSync® and Guidant's Contak™). These cardiac resynchronization systems are implanted under the skin of the chest and connected to three leads (soft insulated wires) that are inserted through veins into the heart. One lead is place in the right atrium, a second in the right ventricle and the third is placed into the coronary sinus or one of its tributaries such as lateral (marginal) or postero-lateral cardiac vein, such that it contacts the outside wall of the left ventricle.

Although not intending to be bound by any particular theory, it is considered that electrical stimulation induces predominant expression of slow fatigue resistant myosin (Chachques et al., 2001, Circulation, 104 (Suppl. 2):555-556, incorporated herein by reference).

In the present invention, the transplanted myoblasts are paced in synchrony with the cardiac cycle by electrostimulation. Pacing requires a sufficient voltage to activate all or most of the transplanted myoblasts. For the present invention, a pulse amplitude of 2.5 to 6 V and a pulse width of 0.2 to 0.7 msec are suitable. In a preferred embodiment, the pulse amplitude is 5 V and the pulse width is 0.5 msec. Similar values can be used for in vitro stimulation of myogenic cells. The pulse rate for the in vitro stimulation can be around 120/min. It will be recognized by those skilled in the art that while exemplary values are provided herein, other values can be determined by those skilled in the art by standard techniques.

In one embodiment, of the invention, the in vitro electrical stimulation of the cultured myogenic cells is combined with multisite cardiac pacing following implantation.

The times for culturing myogenic cells for the present invention and for electrostimulation in vitro can easily be determined by those skilled in the art. In one example, cells can be obtained by muscle biopsy, cultured for about 3 weeks, electrically stimulated for 2 weeks, implanted into the damaged myocardium and electrically stimulated to synchronize.

Cells can also be electrostimulated before implantation in vitro. At least two cell types can benefit from in vitro electrostimulation. 1) Skeletal myoblasts (which undergo increased expression of fatigue resistant slow myosin after 2-week electrostimulation). 2) Bone marrow stromal cells, which are multipotent mesenchymal stem cells that can undergo milieu-dependent differentiation and develop a corresponding phenotype. Marrow stromal cells, when transplanted in normal myocardium, can become cardiocytes. The role of electrostimulation is to “pre-condition” stem cells for pre-differentiation into myogenic cells before myocardial implantation.

### EXAMPLE 1

This embodiment describes the isolation of skeletal myoblasts.

Skeletal Muscle Biopsy

A 1 cm³ skeletal muscle piece (6-8 grams) was explanted from the patient's leg or arm, under sterile conditions. The biopsy was kept in Hank's Balanced Salt Solution (Gibco) at 4° C. until cell culture started. The operative wound was then closed.

Satellite Cell Isolation and Culture

The explanted skeletal muscle pieces were washed in phosphate buffered saline (PBS, Gibco). In a Petri dish, adipose tissue and fascia were removed and the muscle was minced with scissors. The muscle fragments were washed in PBS until the supernatant remained clear. Centrifugation (Sigma 3K10, Bioblock) was carried out at 300 g for 5 minutes. The PBS was replaced with 20 mL of 0.25% trypsin-EDTA (Gibco) and placed in a 37° C. shaking waterbath. After 40 minutes the fragments were forced through a 10 mL disposable pipette. Following aspiration, cells were filtered through a 40 g nylon cell strainer (Polylabo). The remaining muscle fragments on the filter were again subjected to enzymatic and mechanical digestion.

One mL of fetal calf serum (Gibco) was added to the filtrate and the solution was centrifuged at 300 g for 20 minutes. The resulting cell pellets were pooled in 10 ml fresh complete culture medium: 79% Ham-F12 medium, 25 pg/ml bFGF (human recombinant, Sigma), 20% Fetal Calf Serum, 1% penicillin/streptomycin (Gibco) and plated in a 100 mm Petri dish. Cell cultures were incubated at 37° C. in a humidified atmosphere containing 5% CO₂. Passaging of the cultures (1:5 split) was carried out at subconfluency to avoid the occurrence of myogenic differentiation at higher densities. During the first passage, pre-plating was applied to remove fibroblasts which attach quicker than satellite cells. The satellite cells are implanted upon the third passage.

The number of satellite cells in the primary culture was determined using immunofluorescence with a desmin primary antibody (1:20 Sigma) followed by FluoroLink™ Cy™3 (1:200, Amersham Pharmacia Biotech) as a second antibody.

Satellite Cell Preparation

Before cell implantation, the growth medium of each satellite cell culture was tested aerobically and anaerobically in broth for its sterility. The Petri dishes (100 mm) containing about 1 million cells each were washed with PBS. Upon detachment of the cells using 2 mL trypsin-EDTA, 2 mL of complete culture medium were added to each cell suspension. The contents of the 100 culture dishes were pooled and spun at 300 g for 15 minutes. The supernatant was removed and replaced with 20 mL of PBS. The cell concentration and viability were determined with Trypan blue (Gibco) using a Malassez cytometer (Polylabo). The calculated volume of cell suspension containing 100 million cells (or more, up to 800 million cells) is transferred to a 50 mL tube and centrifuged at 300 g for 5 minutes. The final cell pellet was resuspended in 1 ml of 0.5% bovine serum albumin (BSA, Sigma) diluted in Ham-F12 culture medium.

Satellite Cell Implantation

Satellite cells were injected in the ventricular lesion. The heart was exposed by minithoracotomy or sternotomy. The infarction site was identified. Satellite cells were then injected using a Hamilton syringe, by multiple injection points (5 to 10). The number of implanted cells, the volume of injection and the number of injection points depend on the size and the configuration of the myocardial infarcted area. Injections can be epicardial using standard or minimally invasive thorascopic procedures, endoventricular using a catheter based cell delivery assisted by 3D electromechanical mapping bi-plane fluoroscopy and ultrasound guidance and/or with an MRI compatible catheter, or intravascular by catheter based intracoronary, intravenous, or systemic methods.

Electrostimulation

In an experimental the possibility of electrostimulating the ventricles following autologous skeletal cell implantation as a potential therapeutic option for heart failure was explored. The goal of electrostimulation was to transforming passive cell therapy into an active ventricular assist procedure.

Electrostimulated transplanted myoblasts survived and improved ventricular function of infarcted hearts. Cell distribution and development of myotubes into the myocardium were improved by associating cell therapy with cardiac pacing. Electrostimulatation enhanced myoblast contractile activity which promotes the slow myosin heavy chain (MHC) expression, better adapted to perform a cardiac work.

### EXAMPLE 2

This embodiment demonstrates that electrical stimulation of implanted myogenic cells improves heart function. To illustrate this embodiment, the following groups were used. Group 1 (n=6): Infarction (control). Group 2 (n=6): Infarction+atrial synchronized biventricular pacing (BV) stimulation (control). Group 3 (n=5): Infarction+myoblast transplantation. Group 4 (n=5) Infarction+myoblast transplantation+BV. Groups 3 and 4 included 25 million cultured myoblasts (from femoral biceps muscle) labeled with DAPI (diamidino phenylidolo) and were injected in the infarcted area. In groups 2 and 4, BV was performed using epicardial electrodes. Serum troponin I levels were used to evaluate the infarction. Echocardiographic and immuno-histological studies were performed at 2 months. Two animals died after infarction. Serum troponin I rose to 126+/−70 ng/ml 2 days following infarction. Echocardiography showed a significant improvement in ejection fraction (47+/−vs 36+/−4%) and a limitation of LV dilation (49+/−7 vs 69+/−2 ml) in group 4 vs control group. Viable DAPI labeled cells were identified in the infarcted areas. Differentiation of myoblasts into myotubes was significantly improved in group 4. In this group, immunocytological studies showed enhanced expression of slow myosin heavy chain compared to other groups. These results demonstrate that electrostimulation enhanced expression of slow myosin heavy chain which is better adapted at performing cardiac work.

### EXAMPLE 3

The in vitro electrostimulation can be carried out in culture flasks/dishes. An example is presented as follows. Single chamber bipolar pacemakers and 2 temporary cardiac leads (having a platinum-iridium alloy in the electrode surface) for each culture flask, are used to electrostimulate the cell culture, medium and cells. Both sterile electrodes (cathode and anode) are submerged separated into the flasks. Chronic electrostimulation starts 7 days after cell seeding. Single bipolar pulses with a pulse amplitude of 5 Volts, pulse width of 0.5 milliseconds, at a rate of 120 pulses per minute are delivered. Stimulation lasted for up to 14 days until the cells were harvested for myocardial implantation. During the cell culture process, different passages are done in order to obtain the final cells quantity. At each passage the cell suspension is split in 5 other flasks. After 3 weeks, more than 200 million cells are obtained. Two electrodes are used for each tissue culture flask of 300 cm², and one pacemaker can be coupled to 10 electrodes using special connectors. The rationale to pace the cell cultures at a rate of 120 per minute is to imitate the fetal heart rate in order to physiologically promote myogenic cell differentiation. Implantation is then carried out as in Example 1.

### REFERENCES

1) Chachques et al. Progress in Artificial Organs, Cleveland, Ohio, 1986, ISAO Press, P 409-412.

2) Chachques et al. Ann NY Acad Sciences 1987, 494: 445-448.

3) Chachques et al. Circulation 1988, 78 (Suppl 3): 203-216.

4) Rajnoch et al. J Thorac Cardiovasc Surg (in press).

5) Salmons et al. Muscle & Nerve 1981; 4:94-105.

6) Salmons et al. J. Physiol. Lond. 1969, 210:535-549.

7) Salmons et al. Nature 1976; 263: 30-34.

8) Pette et al. Pfluegers Arch 1973; 338: 257-272.

9) Naumann et al. Differentiation 1994; 55: 203-211.

10) Wehrle et al. Differentiation 1994; 58: 37-46.

11) Pette et al. Rev Physiol Biochem Pharmacol 1992; 120: 116-202.

12) Taylor et al. Nat Med 1998; 4: 929-933.

13) Dorfman et al. J Thorac Cardiovasc Surg 1998; 116: 744-751.

14) Scorsin et al. J Thorac Cardiovasc Surg 2000; 119: 1169-75.

15) Gras et al. Pace 1998; 21 (Pt.II): 2249-2255.

16) Auricchio et al. Circulation 1999; 99: 2993-3001.

17) Stellbrink et al., 2001, J. Am. Coll. Cardiol. 38:1957-65

18) orlic et al., 2001, Nature, 410:701-705

19) tomita et al., 1999, Circulation, 100:247-56

What is claimed is:

1. A method for repairing the myocardium of an individual comprising the steps of:
a. obtaining myogenic cells;
b. implanting said myogenic cells into the damaged myocardium; and
c. providing electrical stimulation to the implanted cells to induce the implanted cells to contract in synchrony with the surrounding tissue.

2. The method of claim 1, wherein the myogenic cells are selected from the group consisting of skeletal myoblasts, bone marrow stromal cells, peripheral blood stem cells, post natal marrow mesodermal progenitor cells, smooth muscle cells, adult cardiomyocytes, fetal cardiomyocytes, neonatal cardiomyocytes, embryonic stem cells, cell lines, bone marrow derived angioblasts, endothelial cells, endothelial progenitor cells and combinations thereof

3. The method of claim 1, wherein the electrical stimulation comprises of at least one pulse having an amplitude between 2 to 6 volts and a width of 0.2 to 0.7 msec.

4. The method of claim 3, wherein the pulse has an amplitude of 5 volts and a width of 0.5 msec.

5. A method for repairing the myocardium of an individual comprising the steps of:
a. obtaining myogenic cells;
b. proliferating the myogenic cells in culture;
c. electrically stimulating the myogenic cells in culture; and
d. implanting the electrically stimulated myogenic cells into the damaged myocardium;

6. The method of claim 5, further comprising the step of providing electrical stimulation to the implanted cells to induce the implanted cells to contract in synchrony with the surrounding tissue.
